(19) United States
(12) Patent Application Publication
(10) Pub. No.: US 2015/0166952 A1
(43) Pub. Date: Jun. 18, 2015

(54) CULTURED HEPATOCYTE AND METHOD FOR PREPARING THE SAME

(71) Applicant: Industrial Technology Research Institute, Hsinchu (TW)

(72) Inventors: Chen-Ming Chen, Nantou County (TW); Wannhsin Chen, Hsinchu City (TW); Lih-Tao Hsu, Hsinchu County (TW)

(73) Assignee: Industrial Technology Research Institute, Hsinchu (TW)

(21) Appl. No.: 14/106,874

(22) Filed: Dec. 16, 2013

(51) Int. Cl. C12N 5/071; G01N 33/50

(52) U.S. Cl. CPC C12N 5/067; G01N 33/5014; G01N 33/5008

(57) ABSTRACT

Cultured hepatocytes with hepatic-cord structure and the applications were disclosed. Also the disclosure performed the method for obtaining the cultured hepatocytes with hepatic-cord structure from pluripotent stem cells and progenitor cells.

### BACKGROUND

1. Technical Field

The technical field relates to a cultured hepatocyte and a method for preparing the same.

2. Background

The pharmaceutical industry has an unmet need for human hepatocytes to pre-clinically evaluate new drug hepatotoxicity. However, the supply of primary human hepatocytes is insufficient due to the competing demand for livers for orthotopic liver transplantation. The quality of primary human hepatocytes is also varying and donor dependent, and they rapidly lose their functional properties when used for applications in vitro. Although animal models and transformed human cell lines are also used to assess drug metabolism and toxicities, they are not fully reliable predictors of normal human response and often fail to hinder weak lead candidates to enter clinical phases. Many drugs found to be responsible for liver injury during clinical trials did not cause any liver damage in animal experiments. Therefore, alternative source of human hepatocytes is of the greatest interest by the pharmaceutical industry today. The human hepatocytes are currently the FDA golden standard for evaluation of drug hepatotoxicity.

The human hepatocytes for evaluation of drug are needed.

### SUMMARY

One embodiment of the disclosure provides a cultured hepatocyte derived from pluripotent stem cells, which has a hepatic cord-like structure morphology in vitro.

One embodiment of the disclosure provides a method for preparing a hepatocyte, which comprises the following steps: providing a pluripotent stem cell, differentiating the pluripotent stem cell into a progenitor cell, proliferating the progenitor cell, and inducing the progenitor cell into the hepatocyte having a hepatic cord-like structure morphology.

One embodiment of the disclosure provides a method for preparing a hepatocyte, which comprises the following steps: providing a progenitor cell, proliferating the progenitor cell, and inducing the progenitor cell into the hepatocyte having a hepatic cord-like structure morphology.

One embodiment of the disclosure provides a method for screening an agent, which comprises the following steps: providing the cultured hepatocyte having a hepatic cord-like structure morphology, treating the cultured hepatocyte with an interest, and determining the interest to be the agent or not.

Several exemplary embodiments accompanied with figures are described in detail below to further describe the disclosure in details.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings are included to provide a further understanding of the invention, and are incorporated in and constitute a part of this specification. The drawings illustrate embodiments of the invention and, together with the description, serve to explain the principles of the invention.

FIG. 1 shows the hepatic cord structure morphology of cultured hepatocytes derived from TW6 human embryonic stem cells (hESCs) (A) or ITRI-01 induced pluripotent stem cells (iPSC) (B) using the induction protocol, according to one example. The scale bar in the lower right corner corresponds to 100 μm.

FIG. 2 shows the functionality of multidrug resistance-associated proteins 2 (MRP2) transport protein in TW6 hESC (A) or ITRI-01 iPSC (B) derived cultured hepatocytes, as detected by 5(6)-Carboxy-2′,7′-dichlorofluorescein diacetate (carboxy-DCFDA), according to one example. MRP2 is expressed in the canalicular (apical) part of the hepatocyte and functions in biliary transport.

FIG. 3 shows the marker expression in TW6 hESC-derived cultured hepatocytes according to one example. The panels show expression of albumin (ALB), hepatocyte nuclear factor 4 alpha (HNF4A), alpha-1-antitrypsin (AAT), glucose-6-phosphate (G6P), asialoglycoprotein receptor (ASGR2), cytokeratin-18 (CK18), multidrug resistance-associated protein 2 (MRP2) and CYP3A4.

FIG. 4 shows the marker expression in ITRI-01 iPSC-derived cultured hepatocytes according to one example. The panels show expression of albumin (ALB), hepatocyte nuclear factor 4 alpha (HNF4A), alpha-1-antitrypsin (AAT), glucose-6-phosphate (G6P), asialoglycoprotein receptor (ASGR2), cytokeratin-18 (CK18), multidrug resistance-associated protein 2 (MRP2) and CYP3A4.

FIG. 5 shows the result of albumin production by TW6 hESC-derived cultured hepatocytes using the induction protocol, hepaRG cells, and primary human hepatocytes, according to one example.

FIG. 6 shows the CYP3A4 enzyme induction by rifampicin in TW6 hESC-derived cultured hepatocytes, according to one example.

FIG. 7 shows LDL uptake of TW6 hESC (A) or ITRI-01 iPSC (B) derived cultured hepatocytes using DiI-labeled acetylated LDL.

FIG. 8 shows lipid droplets in TW6 hESC or ITRI-01 iPSC derived cultured hepatocytes using Oil red O staining. The scale bar in the lower right corner corresponds to 100 μm.

FIG. 9 shows glycogen accumulation in TW6 hESC or ITRI-01 iPSC derived cultured hepatocytes using PAS staining. The scale bar in the lower right corner corresponds to 100 μm.

FIG. 10 shows troglitazone-induced cytotoxicity in TW6 hESC-derived cultured hepatocytes according to one example.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein.

One embodiment of the disclosure provides a cultured hepatocyte derived from pluripotent stem cell, which has a hepatic cord-like structure morphology in vitro.

The term “hepatocyte” as used herein refers to a cell that has characteristics of epithelial cells obtained from liver, for example cells that express asialoglycoproteinreceptor (ASGR), alpha-1-antitrypsin (A1AT), albumin, hepatocyte nuclear factors (HNF1 and HNF4) and CYP genes (1A1, 1A2, 2B6, 2C8, 2C9, 2D6, 3A4). Other markers of interest for hepatocytes include glucose-6-phosphatase, transferrin, CK18, gamma.-glutamyltransferase, HNF 1.beta., HNF 3.alpha., HNF-4.alpha., transthyretin, CFTR, apoE, glucokinase, insulin growth factors (IGF) 1 and 2, IGF-1 receptor, insulin receptor, leptin, apoAJI, apoB, apoCIII, apoCII, aldolase B, phenylalanine hydroxylase, L-type fatty acid binding protein, transferrin, retinol binding protein, erythropoietin (EPO), transporter proteins, such as multidrug resistance-associated protein 2 (Mrp2) and bile salt export pump (BSEP), and clotting factors, such as Factor V, VII, VIII, IX and X.

Hepatocytes may also have the following biological activities, as evidenced by functional assays. The cells may have a positive response to dibenzylfluorescein (DBF); have the ability to metabolize certain drugs, e.g., dextromethorphan and coumarin; have drug efflux pump activities (e.g., P glycoprotein, Mrp2 activity); upregulation of CYP activity by phenobarbital, as measured, e.g., with the pentoxyresorufin (PROD) assay, which is seen only in hepatocytes and not in other cells (see, e.g., Schwartz et al. (2002) J. Clin. Invest. 109:1291); CYP enzyme induction e.g. CYP3A4 by rifampicin, as determined, e.g., by CYP3A4/Luciferin-IPA assay (see, e.g., Doshi U and Li A P. 2011. Luciferin IPA-based higher throughput human hepatocyte screening assays for CYP3A4 inhibition and induction. Journal of Biomolecular Screening. 16(8): 903-909.); take up LDL, e.g., DiI-acil-LDL (see, e.g., Schwartz et al., supra); store lipids, as determined, e.g., by using Oil red O staining (see, e.g., Osawa Y et al., 2011 Acid sphingomyelinase regulates glucose and lipid metabolism in hepatocytes through AKT activation and AMP-activated protein kinase suppression. 25(4): 1133-1144.); store glycogen, as determined, e.g., by using a periodic acid-Schiff (PAS) staining of the cells (see, e.g., Schwartz et al., supra); produce urea and albumin (see, e.g., Schwartz et al., supra); and present evidence of glucose-6-phosphatase activity. In one example, the cultured hepatocytes have Mrp2 transporter function. In another example, the cultured hepatocytes uptaked LDL and accumulated glycogen and lipids. In yet another example, the cultured hepatocytes expressed at least one marker selected from the group consisting of albumin, HNF4α, AAT, G6P, ASGR2, Mrp2, CK18, and CYP3A4.

The term “membrane polarity” as used herein refers to a property of hepatocytes having distinct apical and basolateral domains. Specific transport mechanisms and receptors are localized to the apical membrane that faces the canalicular lumen (e.g., P-glycoprotein, BSEP, BCRP, MRP2) and the basolateral membrane that faces the pericellular space between hepatocytes and the blood-filled sinusoid (e.g., NTCP, OATP1B1, OATP1B3, OATP2B1, OAT2, OAT7, OCT1, MRP3, MRP4, MRP6). Hepatocytes have membrane polarity, as determined, e.g. by monitoring the expression and function of apical efflux transporters such as Mrp2 using 5(6)-Carboxy-2′,7′-dichlorofluorescein diacetate (carboxy-DCFDA) (see e.g., Goral V N et al. 2010. Perfusion-based microfluidic device for three-dimensional dynamic primary human hepatocyte cell culture in the absence of biological or synthetic matrices or coagulants. Lab Chip 10:3380-3386.). Carboxy-DCFDA is passively absorbed by the hepatocytes, metabolized and fluorescein diacetate is actively effluxed via Mrp2 transport protein into bile canaliculi. This function is driven by restoration of cell membrane polarity. The term “hepatic cord structure morphology” as used herein refers to in vivo hepatocyte structural morphology consisting of a mass of cells, arranged in irregular radiating columns and plates, spreading outward from the central vein of the hepatic lobule. The cells are multi-sided and contain one or sometimes multiple distinct nuclei. Many such cords join to form the parenchyma of the liver lobule. Each cell usually contains granules and some protoplasmic and others consisting of glycogen, fat, or an iron compound.

The term “hepatic cord-like structure morphology” as used herein refers to cultured hepatocytes with structure morphology similar to hepatic cords.

In one example, the cultured hepatocyte had a hepatic cord-like structure morphology in vitro.

One embodiment of the disclosure provides a method for preparing a hepatocyte, which comprises the following steps: providing a pluripotent stem cell, differentiating the pluripotent stem cell into a progenitor cell, proliferating the progenitor cell, and inducing the progenitor cell into the hepatocyte having hepatic cord-like structure morphology.

The term “pluripotent stem cell” as used herein refers to one of the cells that are self-replicating, is derived from human embryos, human fetal tissue or reprogrammed cells and is known to develop into cells and tissues of the three primary germ layers. Although pluripotent stem cells may be derived from embryos, fetal tissue or reprogrammed cells, such stem cells are not themselves embryos. “Self-replicating” means the cell can divide and to form cells indistinguishable from it. The “three primary germ layers”—called the ectoderm, mesoderm, and endoderm—are the primary layers of cells in the embryo from which all tissues and organs develop. Pluripotent stem cells are also known as embryonic stem cells or induced pluripotent stem cells.

According to one embodiment, the pluripotent stem cell could be a human pluripotent stem cell (hPSCs). In another example, the pluripotent stem cells were human induced pluripotent stem cell (iPSC).

As used herein, the term “differentiate” refers to the production of a cell type that is more differentiated than the cell type from which it is derived. The term therefore encompasses cell types that are partially and terminally differentiated. For example, differentiated cells derived from hESC cells are generally referred to as hESC-derived cells or hESC-derived cell aggregate cultures, or hESC-derived single cell suspensions, or hESC-derived cell adherent cultures and the like.

According to one embodiment, the human pluripotent stem cells (hPSCs) were cultured in a suitable medium. The suitable culture medium for human pluripotent stem cells could refer to Thomson J A et al. 1998. Embryonic stem cell lines derived from human blastocysts. Science 282(5391):1145-1147, but not limit thereto. In one example, the culture medium comprises mouse embryonic fibroblasts (MEF), DMEM/F12 medium (Invitrogen Corp) supplemented with 15% knockout serum replacement (Invitrogen Corp), 1 mmol L-glutamine (Invitrogen Corp), 0.1 mmol β-mercaptoethanol, 0.1 mmol NEAA, and 4 ng/ml FGF2.

As used herein, the term “progenitor cell” refers to a biological cell that, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. Controversy about the exact definition remains and the concept is still evolving.

According to one embodiment, the human pluripotent stem cells were differentiated into a hepatic progenitor cell. The methods for differentiation of pluripotent stem cells into hepatic progenitor cells could refer to (1) Tayeb K et al. 2010. Highly efficient generation of human hepatocyte-like cells from induced pluripotent stem cells. Hepatology 51:297-305. (2) Touboul T et al. 2010. Generation of functional hepatocytes from human embryonic stem cells under chemically defined conditions that recapitulate liver development. Hepatology 51:1754-1765. (3) Song Z et al. 2009. Efficient generation of hepatocyte-like cells from human induced pluripotent stem cells. Cell Research 19:1233-1242. (4) Hannan N et al. 2013. Production of hepatocyte-like cells from human pluripotent stem cells. Nature Protocol 8(2):430-437, but not limit thereto. In one example, the cultured hESCs were transferred to a 2% collagen type 1 coated plate and maintained in MEF conditioned medium for 24-48 hours. When cells reached 40-50% confluence, medium was replaced by an endoderm induction medium for 3 days consisting of RPMI medium supplemented with 2% serum replacement and Activin A (100 ng/ml). For hepatic progenitor differentiation, cells were cultured in RPMI medium supplemented with 1% B27, FGF4 (10 ng/ml; Peprotech) and HGF (10 ng/ml) for 5 days. The cells were then split with trypsin and re-seeded at a density of 1-5×10⁴ cells/cm² on collagen type 1-coated plates in DMEM medium supplemented with HGF (20 ng/ml; Peprotech) for 5 days.

The term “proliferate” as used herein refers to cell growth, which is used in the contexts of cell development and cell division (reproduction). When used in the context of cell division, it refers to growth of cell populations.

According to one embodiment, the progenitor cells were proliferated in a specific condition for a period. In one example, the hepatic progenitor cells were cultured and maintained for 5 days to 28 days. In another example, the period for progenitor proliferation also could be 7 days to 14 days. In this period, the hepatic progenitor cells could grow up from the cell density of 1×10⁴ cells/cm² to 1×10⁵ cells/cm². In one example, the hepatic progenitor cells were cultured and maintained in a medium, which comprises DMEM/F12 medium supplemented with 1-10 uM nicotinamide, lx insulin-transferrin-selenium (ITS), 0.1-10 uM dexamethasone, 1-10% human serum albumin, 1-40 ng/ml HGF, 1-40 ng/ml FGF1 and 1-50 ng/ml EGF.

According to one embodiment, the next step after the proliferation of hepatic progenitor cells is inducing the hepatic progenitor cells into hepatocytes.

In one example, the hepatic progenitor cells were cultured in Corning hepatocyte medium supplemented with 0.5-2% DMSO, 0.1-10 uM dexamethasone, 10-100 ng/ml oncostatin M (OSM), and 10-100 ng/ml HGF. The progenitor cells were also overlaid with 1-10% Matrigel. During the differentiation process (from hepatic progenitor into hepatocyte), medium was changed three times a week.

According to one embodiment, the cultured hepatocytes have a hepatic cord-like structure morphology and exhibit MRP2 transporter activities. Thus, the cultured hepatocytes exhibit membrane polarity in vitro. On contrary, pluripotent stem cell-derived hepatocytes without hepatic cord-like structure morphology do not show or show limited membrane polarity in vitro. The cultured hepatocytes of the disclosure are much similar to in vivo hepatocytes, as well as the biological properties thereof is more similar to that of in vivo hepatocytes.

One embodiment of the disclosure provides a method for preparing a hepatocyte, which comprises the following steps: providing a progenitor cell, proliferating the progenitor cell, and inducing the progenitor cell into the hepatocyte having a hepatic cord-like structure morphology.

According to one embodiment, the cultured hepatocyte with hepatic cord-like structure morphology could be derived from a progenitor cell. The steps of proliferating and inducing of progenitor cells have described above.

One embodiment of the disclosure provides a method for screening an agent, which comprises the following steps: providing the cultured hepatocyte having a hepatic cord-like structure morphology, treating the cultured hepatocyte with an interest, and determining the interest to be the agent or not.

According to one embodiment, the above-described cultured hepatocytes were used for evaluating a toxic effect and metabolic product. The cells have hepatic cord-like structure morphology as well as they are good for evaluation of drug hepatotoxicity and metabolized product in vitro.

As used herein, the term “agent” refers to substance with pharmacological or biological activity, i.e., a pharmaceutical drug.

According to one embodiment, an interest to be an agent could be a compound, solvent, protein, nucleic acid, antibody, vaccine, and the like, but not limit thereto. In one embodiment, the agent is troglitazone, for example.

According to one embodiment, the step of treating the cultured hepatocyte with an interest means culturing the cells with adding the interest. It could be adding different amounts of interest as treatment.

According to one embodiment, the method for determining the hepatotoxicity of interest could refer to Fotakis G and Timbrell J A. 2006. In vitro cytotoxicity assays: comparison of LDH, neutral red, MTT and protein assay in hepatoma cell lines following exposure to cadmium chloride. Toxicology Letters. 160(2):171-7, but not limit thereto. In one example, CellTox™ Green Cytotoxicity assay was used for determining the toxic effect.

### EXAMPLES

### Example 1

### Culturing of hESCs and iPSCs

TW6 hESCs were derived from the inner cell mass of an in vitro fertilized human blastocyst. ITRI-01 iPSCs were derived from human foreskin fibroblast cells using lentivirus-mediated delivery of the human factors Oct4, Sox2, Nanog and c-Myc according to manufacturer's instructions (Stemgent Dox inducible reprogramming kit). Both hESCs and iPSCs were cultured and expanded on mouse embryonic fibroblasts (MEF), using DMEM/F12 medium (Invitrogen Corp) supplemented with 15% knockout serum replacement (Invitrogen Corp), 1 mmol L-glutamine (Invitrogen Corp), 0.1 mmol β-mercaptoethanol, 0.1 mmol NEAA, and 4 ng/ml FGF2.

### Example 2

### Differentiating hESCs or iPSCs into Hepatic Progenitor Cells

TW6 hESCs or ITRI-01 iPSCs of example 1 were transferred to a 2% collagen type 1 coated plate and maintained in MEF conditioned medium for 24-48 hours. When cells reached 40-50% confluence, medium was replaced by an endoderm induction medium for 3 days consisting of RPMI medium supplemented with 2% serum replacement and Activin A (100 ng/ml). For hepatic progenitor differentiation, cells were cultured in RPMI/B27 medium supplemented with FGF4 (10 ng/ml; Peprotech) and HGF (10 ng/ml) for 5 days. The cells were then split with trypsin (1:3-1:5) and re-seeded on collagen type 1-coated plates in DMEM medium supplemented with HGF (20 ng/ml; Peprotech) for 5 days.

### Example 3

### Proliferating the Hepatic Progenitor Cells

The hepatic progenitor cells of example 2 were cultured and maintained in the medium, which contains DMEM/F12 medium supplemented with 1×ITS, 10 uM nicotinamide (Sigma-Aldrich), 0.1 uM dexamethasone (Sigma-Aldrich), 10 ng/ml HGF (Peprotech), 10 ng/ml FGF1 (Peprotech), and 10 ng/ml EGF (Peprotech) for 10 days. The cell density in initial culture was 3×10⁴ cells/cm².

### Example 4

### Inducing the Hepatic Progenitor Cells into Hepatocytes

The hepatic progenitor cells of example 3 were then induced for 7 days in another medium, which contains Corning hepatocyte medium supplemented with 0.5% DMSO, 0.1 uM dexamethasone, OSM (100 ng/ml), and 20 ng/ml HGF. The cells were overlaid with 2% Matrigel. During the inducing process, medium was changed three times a week.

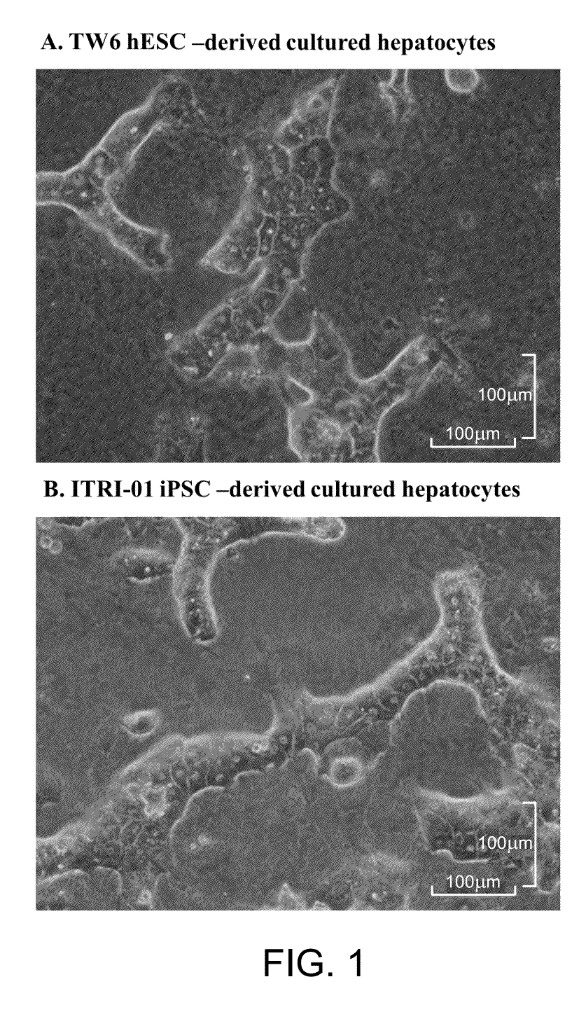

FIG. 1 shows the morphology of the cultured hepatocytes of example 4. As shown in FIG. 1, the cultured hepatocytes derived from TW6 hESCs (A) or ITRI-01 iPSCs (B) were arranged in cords or plates, and this hepatic cord-like structure morphology is similar to in vivo liver tissue.

### Example 5

### Mrp2 Transport Function Assay

The hepatocytes of example 4 were incubated with culture medium containing 5 uM 5(6)-carboxy-2′,7′-Dichlorofluorescein diacetate carboxy-DCFDA; Invitrogen Corp). Carboxy-DCFDA was absorbed by the cells and metabolized. The fluorescent metabolites were actively excreted by Mrp2 transport protein into bile canliculi. After incubation at 37° C. for 1 hr, the cells were washed with PBS to remove the extracellular carboxy-DCFDA. Efflux of fluorescent metabolites was monitored by an inverted fluorescence microscope (Carl Zeiss Microlmaging, Jena, Germany).

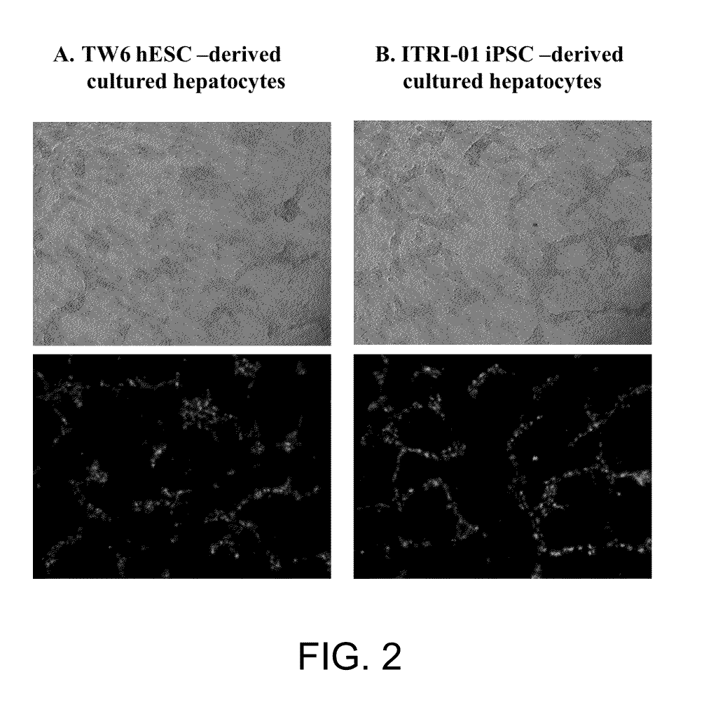

FIG. 2 is the result of Mrp2 transport function of the hepatocytes of example 5. As shown in FIG. 2, the cultured hepatocytes derived from TW6 hESCs (A) or ITRI-01 iPSCs (B) were able to excrete fluorescein diacetate into bile canalicular-like regions between adjacent hepatocytes along the cord-like structures, indicating the presence of membrane polarity in cultured hepatocytes.

### Example 6

### Assay for Hepatocyte Marker Expression

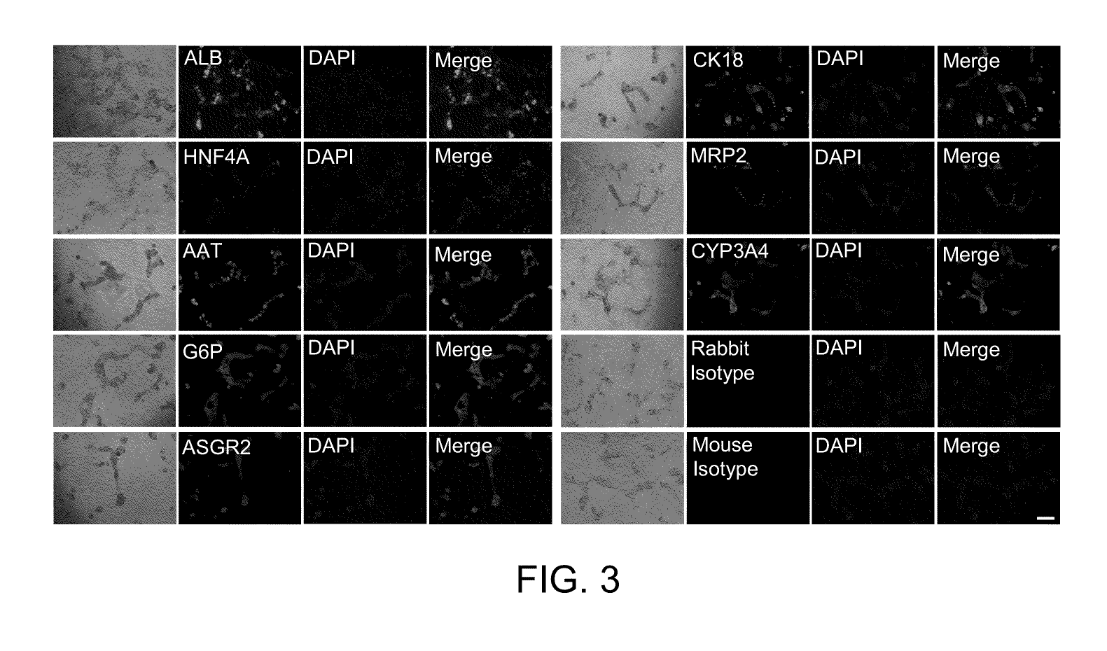

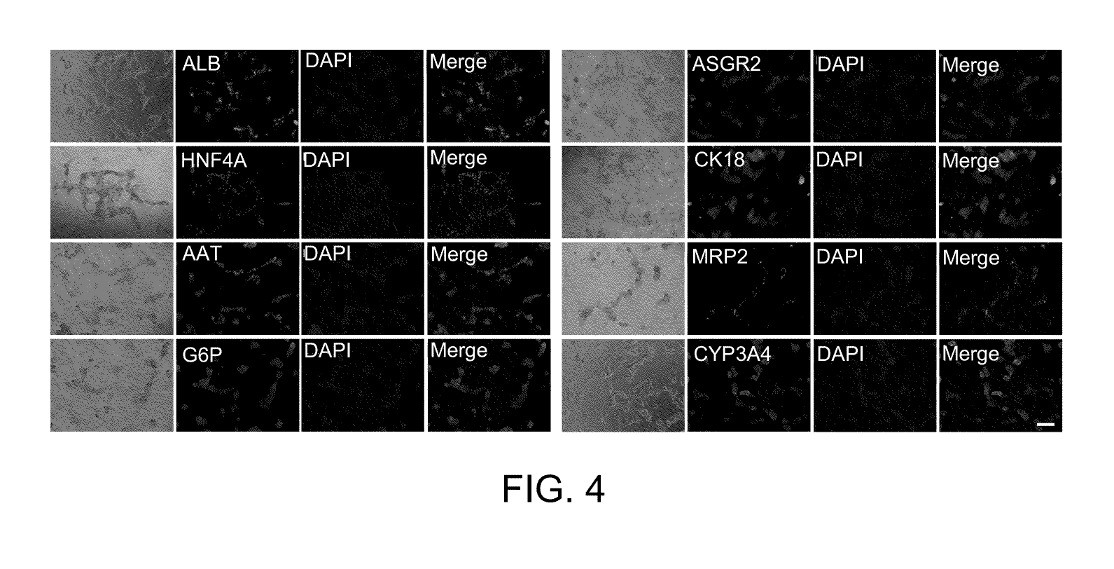

The hepatocytes of example 4 were fixed with 4% paraformaldehyde at 4° C. for 10 min and were then permeabilized in 0.1% Triton-X100 (Sigma-Aldrich) in PBS for 10 min. Fixed cells were washed with PBS and blocked in PBS containing 5% goat serum (Vector) for 1 hr at 4° C., followed by incubating with primary antibodies or isotype controls in PBS containing 1% goat serum at 4° C. overnight. Primary antibodies used were: rabbit anti-human albumin (ALB) conjugated with FITC (1:50; DAKO), mouse anti-human hepatocyte nuclear factor 4 alpha (HNF4a; 1:50; R&D Systems), mouse anti-human alpha1 antitrypsin (AAT; 1:100; abcam), mouse anti-human cytokeratin 18 (Ck18; 1:100; DAKO), rabbit anti-Glucose-6-phosphatase (G6P; 1:100; abcam), rabbit anti-asialoglycoprotein receptor 2 (ASGR2; 1:50; Sigma-Aldrich), mouse anti-human MRP2 (1:100; abcam) and rabbit anti-human CYP3A4 (1:100; abcam). Alexa Fluor 594 anti-mouse IgG, Alexa Fluor 488 anti-rabbit IgG and Alexa Fluor 488 anti-mouse IgG secondary antibodies (Invitrogen Corp.) at a dilution of 1:500 were used for indirect labeling. Cells were counterstained with DAPI (1:10000; Roche Molecular Diagnostics). Fluorescently labeled cells were imaged using an inverted fluorescence microscope (Carl Zeiss MicroImaging). Results were illustrated in FIGS. 3 and 4. As shown in FIGS. 3 and 4, the cultured hepatocytes derived from TW6 hESCs or ITRI-01 iPSCs expressed mature hepatocyte markers including ALB, HNF4a, AAT, Ck18, G6Pase, ASGR2, Mrp2 and CYP3A4.

### Example 7

### Albumin Secretion Assay

The secretion of albumin by the cultured hepatocytes of example 4, hepaRG cells (Invitrogen) and primary human hepatocytes (Lonza) were analysed. HepaRG cells and primary human hepaotcytes were cultured according to manufacturer's instructions. Culture medium was harvested 48 hrs after incubation and assayed for albumin secretion using an enzyme-linked immunosorbent assay (ELISA) kit (Bethyl). Albumin secretion levels were calculated per 10⁵ cells and normalized to time.

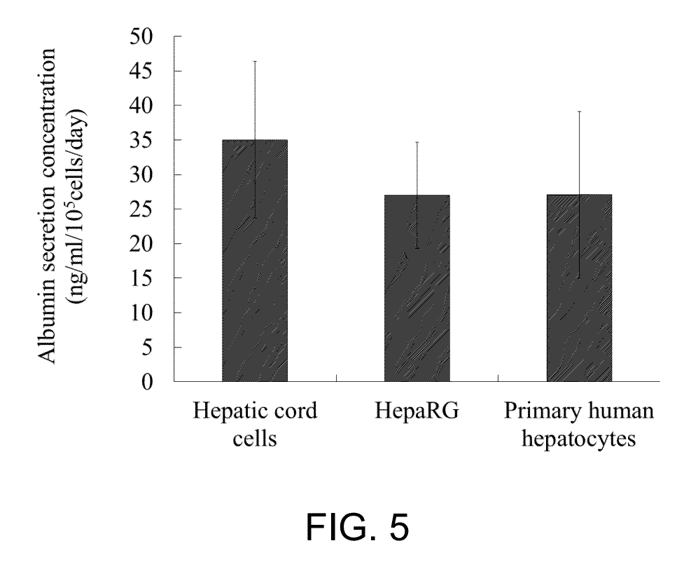

Results were illustrated in FIG. 5. As shown in FIG. 5, the cultured hepatocytes with hepatic cord structure exhibited albumin secretion (35 ng/ml/10⁵ cells/day) at a level comparable to cultured primary human hepatocytes and HepaRG cells.

### Example 8

### CYP3A4 Induction Assay

The hepatocytes of example 4 were incubated with rifampicin (10 μM) in Corning hepatocyte medium to induce CYP3A4 protein levels. Vehicle control wells were incubated with DMSO in Corning hepatocyte medium. After 72 hr incubation, medium was removed and metabolism was determined using P450-Glo CYP3A4 with Luciferin-IPA (Promega) according to the manufacturer's instructions for use. After 60 min incubation with Luciferin-IPA substrate, medium was transferred to an opaque 96-well microtiter plate containing an equal volume of P450-Glo reaction buffer. After 20 min incubation, the luminescent was determined with a plate-reading luminometer (Molecular Devices). For each measurement, wells without substrate were assayed, and values obtained were subtracted from the wells with substrate. These values were then normalized to the cell number. Mean luminescence units per 10⁵ cells in duplicate wells were calculated.

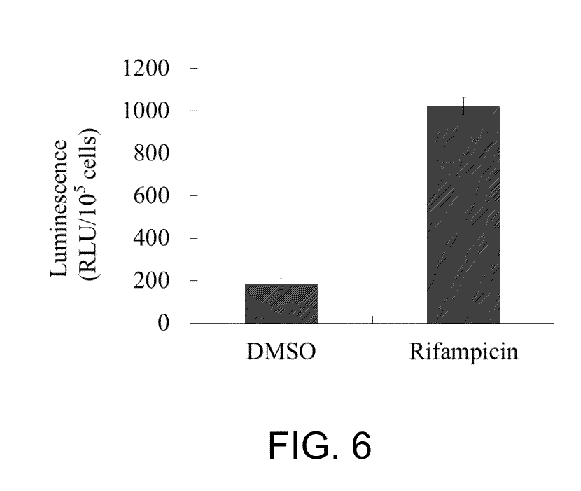

Results were illustrated in FIG. 6. As shown in FIG. 6, the cells exhibited approximately 5 fold induction of CYP3A4 activity after rifampicin induction.

### Example 9

### LDL Uptake, Glycogen and Lipid Accumulation

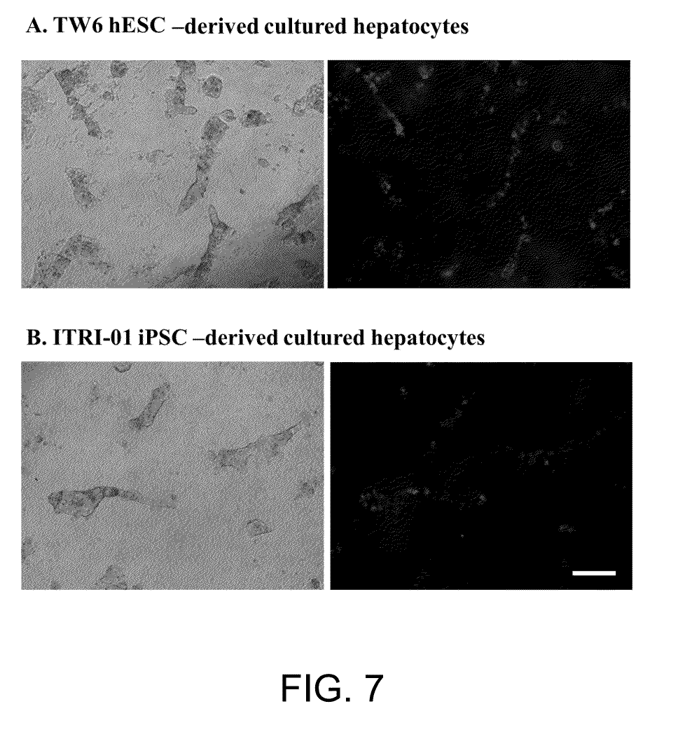

For LDL uptake assay, the hepatocytes of example 4 were incubated with 5 μg/ml of DiI-Ac-LDL (Invitrogen) at 37° C. overnight. After washing with PBS twice, fluorescently labeled cells were imaged using an inverted fluorescence microscope (Carl Zeiss MicroImaging), and results were illustrated in FIG. 7. FIG. 7 shows that the cultured hepatocytes derived from TW6 hESCs or ITRI-01 iPSCs uptaked low density lipoprotein (LDL).

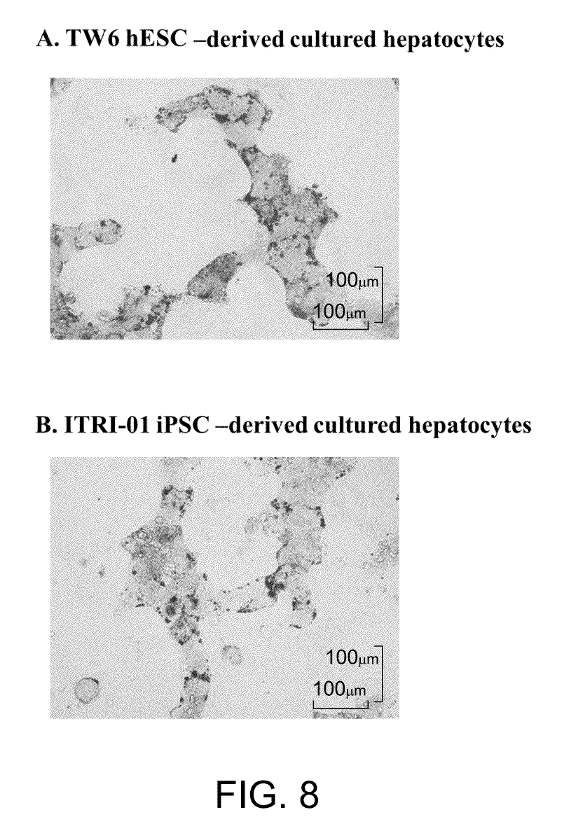

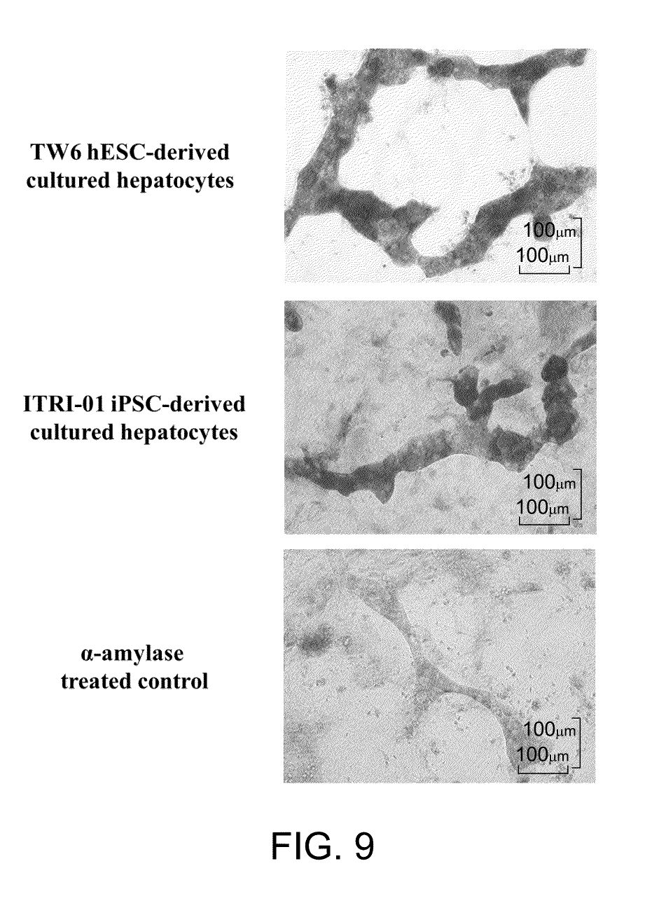

For detection of lipids, the hepatocytes of example 4 were fixed with 4% paraformaldehyde at 4° C. for 10 min and were stained with 0.3% Oil Red O solution (Sigma-Aldrich) for 30 min. The results were illustrated in FIG. 8, which showed lipid droplets at the cell periphery of the cultured hepatocytes derived from TW6 hESCs or ITRI-01 iPSCs. Intracellular glycogen was analyzed by Periodic-acid-Schiff (PAS) staining Cells were fixed with 4% paraformaldehyde at 4° C. for 10 min and oxidized in 0.5% periodic acid for 5 min. After oxidation, cells were rinsed 3 times with distilled water and then treated with Schiff's reagent (Sigma-Aldrich) for 15 min. After the cells were rinsed with distilled water for 5 min, the cells were counterstained with Mayer's hematoxylin for 1 min. Negative control cells were treated with 0.5% alpha-amylase (Sigma-Aldrich) to confirm glycogen. The results were illustrated in FIG. 9, which showed that cultured hepatocytes derived from TW6 hESCs or ITRI-01 iPSCs were able to accumulate glycogen.

### Example 10

### Troglitazone-Induced Hepatotoxicity Assay

The cultured hepatocytes of example 4 were treated with a human hepatoxicant, troglitazone (Sigma-Aldrich) at various concentrations (0, 100, 200, 300, 400 and 500 μM) for 5 days. Cytotoxicity was analyzed using a CellTox™ Green Dye kit (Promega) according to manufacturer's instructions for use. The data was normalized to the non-troglitazone treated control.

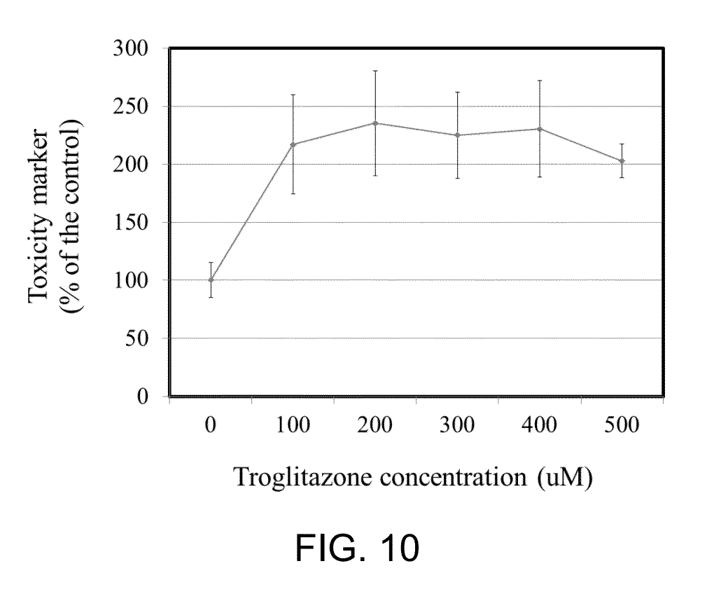

FIG. 10 is the result of troglitazone-induced cytotoxicity in cultured hepatocytes of example 4. As shown in FIG. 10, troglitazone at >100 uM caused an increase in cytotoxicity in the cultured hepatocytes after 5 days of treatment, suggesting that the cultured hepatocytes could be used for evaluation of drug hepatotoxicity.

In contrast to conventional pluripotent stem cell-derived hepatocytes in vitro, the embodiments of the disclosure provide a cultured hepatocyte with hepatic cord-like structures and exhibit Mrp2 transport function, indicative of apicobasal cell polarity, in which is much similar to the liver tissue. The provided cultured hepatocytes could be used in lab and/or clinical drug evaluation, like the drug hepatotoxicity.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A cultured hepatocyte derived from a pluripotent stem cell, which the hepatocyte having a hepatic cord-like structure morphology.

2. The hepatocyte of claim 1, wherein the hepatocyte has Mrp2 transport function and membrane polarity.

3. The hepatocyte of claim 1, wherein the hepatocyte uptakes LDL and accumulates glycogen and lipids.

4. The hepatocyte of claim 1, wherein the hepatocyte expresses at least one marker selected from the group consisting of albumin, HNF4α, CK18, AAT, G6Pase, ASGR2, Mrp2 and CYP3A4.

5. The hepatocyte of claim 1, wherein the hepatocyte is derived from human embryonic stem cell.

6. The hepatocyte of claim 1, wherein the hepatocyte is derived from human induced pluripotent stem cells.

7. A method for preparing a hepatocyte, comprising:
providing a pluripotent stem cell;
differentiating the pluripotent stem cell into a progenitor cell;
proliferating the progenitor cell; and
inducing the progenitor cell into the hepatocyte having a hepatic cord structure morphology.

8. The method of claim 7, wherein the method comprises proliferating the progenitor cell in a medium, comprising DMEM/F12 medium supplemented with 1-10 uM nicotinamide, 1× insulin-transferrin-selenium (ITS), 0.1-10 uM dexamethasone, 1-10% human serum albumin, 10-40 ng/ml HGF, 10-40 ng/ml FGF1 and 10-50 ng/ml EGF.

9. The method of claim 7, wherein the method comprises proliferating the progenitor cell for 5 to 28 days.

10. The method of claim 7, wherein the method comprises proliferating the progenitor cell from the cell density of 1×10⁴ cells/cm² to 1×10⁵ cells/cm².

11. A method for preparing a hepatocyte, comprising:
providing a progenitor cell;
proliferating the progenitor cell; and
inducing the progenitor cell into the hepatocyte having a hepatic cord structure morphology.

12. The method of claim 11, wherein the method comprises proliferating the progenitor cell in a medium, comprising DMEM/F12 medium supplemented with 1-10 uM nicotinamide, 1× insulin-transferrin-selenium (ITS), 0.1-10 uM dexamethasone, 1-10% human serum albumin, 1-40 ng/ml HGF, 1-40 ng/ml FGF1 and 1-50 ng/ml EGF.

13. The method of claim 11, wherein the method comprises proliferating the 15 progenitor cell for 5 to 28 days.

14. The method of claim 11, wherein the method comprises proliferating the progenitor cell from the cell concentration or density of 1×10⁴ cells/cm² to 1×10⁵ cells/cm².

15. A method for screening an agent, comprising:
providing the cultured hepatocyte as claimed of claim 1;
treating the cultured hepatocyte with an interest; and
determining the interest to be the agent or not.

16. The method of claim 15, wherein the method comprises determining a toxic effect of the interest on the cultured hepatocyte.

17. The method of claim 15, wherein the method comprises determining a metabolized product of the interest by the cultured hepatocyte.
